(19) United States
(12) Patent Application Publication
(10) Pub. No.: US 2006/0013822 A1
(43) Pub. Date: Jan. 19, 2006

(54) Utilization of mhc class II binding motifs in immunization to produce immune, serum, monoclonal antibodies and vaccines

(76) Inventors: Thomas V. Tittle, Portland, OR (US); Keith Wegmann, Vancouver, WA (US)

Correspondence Address: FOLEY & LARDNER LLP, 1530 PAGE MILL ROAD, PALO ALTO, CA, 94304, US

(21) Appl. No.: 10/512,325

(22) PCT Filed: Mar. 21, 2003
(86) PCT No.: PCT/US03/08863

§ 371 Date: Jun. 9, 2005

(30) Foreign Application Priority Data
Mar. 22, 2002 (US) 60367042

(51) Int. Cl. A61K 39/00; C12Q 1/68; C07H 21/04; C12P 21/06; C07K 14/74

(52) U.S. Cl. 424/185.1; 435/6; 435/69.1; 435/320.1; 435/325; 530/350; 536/23.5

(57) ABSTRACT

This invention provides compositions and methods for raising humoral antibody responses. The compositions are peptides containing major histocompatibility Class 11 antigen binding motifs (ABM) either native or inserted into the peptide sequence. The ABM can be at the carboxy or amino terminus of the peptide and is shown to provide a T cell epitope thereby assuring adequate T cell help. Associated with the ABM is an extended peptide that rests outside the Class 11 molecule and that is recognized by the B cell, a B cell epitope. This B cell epitope can be a contiguous peptide sequence either at the amino or carboxy terminus. The extended peptide can be irrelevant and can serve as a bridge to an attached B cell epitope such as a hapten. These haptens can be any chemical structure such as a fluorescein molecule or a carbohydrate. The compositions and methods of this invention provide inexpensive vaccines to raise antibodies.

### TECHNICAL FIELD

The invention relates to the field of immunology and in particular to methods of stimulating B cells to produce antibody. This invention is a method to render monomeric antigens immunogenic for B cells. More specifically, this invention relates to the use of an antigenic moiety associated with a peptide containing one or more Major Histocompatibility Complex (MHC) Class II binding motifs as a means to stimulate B cells to produce antibody against the antigenic moiety. Furthermore, immunization with an antigenic moiety associated with a peptide containing one or more MHC Class II binding motifs may serve as a novel method of vaccination.

### BACKGROUND OF THE INVENTION

Induction of an Antibody Response.

The initial signaling events for the activation of B cells and T cells has been known for 20-30 years. B cells have membrane bound receptors with the ability to recognize a single antigen specificity. T lymphocytes have structurally similar receptors that recognize a single small peptide presented in the context of the Major Histocompatibility Complex (MHC) molecules, Class I for CD8+ T cells and Class II for CD4+ T cells. For both B cells and T cells, a signal is delivered during the cross-linking of these antigen receptors.

Two Signals (Signal 1 and Signal 2) are required for induction of B lymphocytes (B cells) to make an immunoglobulin, or antibody molecule. Signal 1 is initiated by recognition of an immunogen by a receptor present on a B cell. The B lymphocyte antigen receptor is an identical copy of the antibody that the cell is prepared to make. Each B cell presents multiple copies of the same receptor and the immunogen is known to contain multiple receptor binding sites or epitopes. These epitopes may be the same or different. These receptors being identical recognize the same antigenic determinant, or epitope.

Signal 1 is received by the B cell when its immunoglobulin receptors are cross-linked by presentation of the multiple copies of the same epitope. This brings together the Iga and Igb signaling molecules and initiates the cascade of molecular signals. After multi-epitopic presentation of an antigen, the B cell is poised to respond to Signal 2. For this reason, highly polymeric antigens, i.e., antigens possessing multiple copies of the same epitope, are very strong inducers of Signal 1 to the B cell. Polymeric antigens are found in protein aggregates, as in carbohydrates whose structure is a repeated sugar moiety or sugars linked in a repeated fashion or as multiply conjugated vaccines. Conversely, mono-epitopic, or haptenic antigens are usually not immunogenic and fail to raise antibody responses. Short peptides and other such small moieties can be made immunogenic by conjugation to large protein carriers, this assures that the moiety is presented in a polymeric fashion leading to the induction of an antibody response.

The process of “Signal 2” starts with internalization of the crosslinked receptor/antigen complexes by the B cell where the antigen undergoes digestion to generate small peptide fragments of the antigen. Once generated, only those peptide fragments that have affinity for MHC Class II molecules can bind to the peptide binding groove of the MHC Class II molecule. The affinity of this interaction is dependent upon the peptide containing MHC Class II binding motif. MHC Class II binding motifs are defined as having characteristic amino acid residues at defined positions. It is these amino acid residues that interact with the MHC Class II molecule to form a stable complex. These MHC Class II/peptide complexes are transferred to the surface of the B cell. This step sets up the B cell for the interaction with a CD4+ helper T lymphocyte (T-cell) that will ultimately deliver the second necessary signal (Signal 2) for the induction of the B cell. Thus, the delivery of multimeric Signal 1 is critical to the activation of the B cell.

Induction of T-Cell Helper Responses.

The T-cell receptor is a member of the Immunoglobulin Superfamily of molecules that share similar structural features. For T-cells, as for B cells, all of the receptors on a given cell recognize the same antigen. However, unlike B cells whose receptors bind to and interact with free antigen, the T-cell receptor recognizes small peptide fragments when they are “presented” in association with the MHC Class II antigen. As with B cells, the crosslinking of multiple T-cell receptors delivers Signal 1 and begins the activation sequence of T-cells. Which in turn, is the critical step in the induction, or activation of the T-cell.

T-Cell Recognition of Antigen: Class II Binding Motifs.

Numerous peptide antigens that certain specific CD4⁺ T-cells recognize have been defined. These empirically defined sequences generally range between 8 and 15 amino acids in length and are usually not recognized by both T cells and B cells. Such peptides have been used as vaccines to raise memory T cells. Most T cell antigens, however, are not defined. Therefore, as with B cells, to ensure that T cell responses occur, immunization with whole protein is the preferred method of inducing T cell help. These proteins are sufficiently large to assure that once digested they contain at least one peptide fragment that can bind to and be presented by the MHC Class II molecule. It is important to note that peptide binding to MHC Class II can occur in situ or they can be loaded externally and compete for binding with in situ loaded peptide.

Recently, the concept of there being specific MHC Class II binding residue motifs within peptide antigenic fragments has been advanced. The importance of Class II binding motifs has been most strongly shown in the recognition of peptides containing the ST/ED motif in the Lewis rat as recently defined. This motif consists of a serine (S) or threonine (T) residue followed by glutamic (E) or aspartic (D) acid residue separated by five intervening amino acids. (S/TXXXXXE/D, wherein X is any amino acid) (SEQ ID NO: 1). The ability of protein sequences containing the ST/ED motif to induce T-cell mediated autoimmune disease in Lewis rats has been amply demonstrated. Of the sixteen or so putative motif sequences for mice with the MHC Class II molecule designated I-A^{b} (such as C57b1/6 mice and their congeners), only one has been systematically demonstrated to be valid through experimentation. This motif is a Phenylalanine (F) followed by any seven residues ending in either an Alanine (A) or an 30 Asparagine (N) residue (FXXXXXXXY, wherein X is any amino acid and Y is A or N (SEQ ID NO: 2).

Current Methods for Raising Antibodies.

Attenuated, live bacterial vaccines were shown by Louis Pasteur to provide protective humoral immunity. The growth and production costs for this type of vaccine are not insignificant but the most widely recognized problem is the concern over safety because of incomplete attenuation. As a result, immunologists have focused much attention on the identification of antigens that are expressed by pathogens and which convey protective humoral immunity. These antigens can then be purified and used as vaccines rather than the attenuated organisms. The antigens that typically provide protective humoral antibody responses are proteins or polysaccharides.

Raising antibodies to proteins for vaccines requires purification of the protein and in some cases, as for toxin molecules, inactivation of the protein's function. This is not always possible either because of low available quantities of protein or because of the vagaries of the purification procedure itself. Inactivating or toxoiding a protein has problems with the incomplete inactivation, although use of mutant gene products called CRM's (for cross reactive material) that lack function makes this less of a problem. Raising antibodies to specific proteins for commercial use other than as vaccines, for example to newly discovered proteins, is also of value. Such antibodies are of interest for the analyses of protein expression and structure.

Anti-carbohydrate antibodies represent another case where carrier-specific help is recognized as playing an important role in the generation of antibodies (Cellular and Molecular Immunology, A. Abbas, A. Lichtman and J. Pobor eds., W. B. Saunders Co. 4^{th} edition, 2000). Anti-polysaccharide antibodies are known to provide protective immunity to bacterial pathogens in adults. These highly polymeric antigens generate good responses of the IgM class but with little IgG being elicited. They are inefficient inducers of B cell memory. Newborns and infants, however, are not capable of responding to these antigens. For this reason, current vaccines for children against, for example, Hemophilus influenzae type b polysaccharide, are conjugate vaccines in which the bacterial polysaccharide is chemically conjugated to a carrier protein such as Diphtheria toxoid. This not only renders them immunogenic in children but also induces isotype switching, such that IgM and IgG isotypes are produced. Memory B cells are also produced. As mentioned above, conjugation ratios are important and need to be monitored. Also, for human injections there are few suitable carriers identified.

Recently, peptide mimics of polysaccharides have been identified using specific anti-polysaccharide antibodies and peptide phage display libraries. Such mimics have been demonstrated for 1) Streptococcus pneumoniae serotype 4 capsular polysaccharide (Lesenski et al., (2001), Vaccine 19:1717-1726), 2) Neisseria meningitidis outer membrane Lipooligosaccharide (Charalambous and Feavers (2000) FEMS Microbiology Letters 191:45-50), and 3) the fucosyl □1-3-N-acetyl-glucosamine linkage of the Lewis Y antigen (Luo et al., (2000), J Biol. Chem. 275:16146-16154) to name but a few. The utility of using these peptide mimics as immunogens requires that they be rendered multimeric by incorporation into lipid proteosomes, by synthesis using polylysine as the backbone to facilitate branching, or by conjugation with a protein carrier, respectively. Thus, the use of these mimics as immunogens will necessitate some form of polymerization or conjugation and will have similar problems as described above for use in manmmals.

Peptides are not currently used as vaccines for humoral immunity. Antibodies to small peptides are difficult to attain in mammals with any predictability. For this reason, it is recommended that peptides be conjugated to larger “carrier” molecules, e.g., keyhole limpet hemocyanin, in order to render them immunogenic (Antibodies: From Design To Assay, 1996, PE Applied Biosystems). The use of such carrier molecules provides multivalent epitope presentation to the B cell for Signal 1 as well as T cell epitopes required for Signal 2 delivery to the B cell. The major concern for all conjugate vaccines is that the ratio of peptide to carrier is critical for immunogenicity with 10 “haptenic” groups per 100 kilodaltons of carrier being optimal. Lesser or greater degrees of conjugation leads to decreased immunogenicity.

To be able to raise specific antibodies to protective bacterial antigens as in vaccines or to specific proteins is clearly a desired commercial endeavor. The synthesis of peptides containing Class II binding motifs is inexpensive and highly purified preparations are easily obtainable. Furthermore, conjugation of haptenic groups can be done during peptide synthesis and most peptide molecules will be conjugated at essentially a 1:1 ratio. Polymeric presentation occurs on the surface of MHC Class II positive cells.

### DETAILED DESCRIPTION OF THE INVENTION

This invention provides the minimal components for raising humoral immunity, or an antibody response, to antigens. The first is an MHC Class II binding motif to stimulate T cells, i.e., a T cell epitope. The second component is a B cell epitope that extends outside of the MHC Class II binding groove so that it is accessible to the B cell receptor.

### BRIEF DESCRIPTION OF THE SEQUENCE LISTINGS

SEQ ID NO: 1 is an amino acid representing an MHC Class II binding motif. This motif consists of a serine (S) or threonine (T) residue followed by glutamic (E) or aspartic (D) acid residue separated by five intervening amino acids. (S/TXXXXXE/D, wherein X is any amino acid).

SEQ ID NO: 2 is an amino acid representing a putative motif sequences for mice with the MHC Class II molecule designated I-A^{b} (such as C57b1/6 mice and their congeners). This motif is a Phenylalanine (F) followed by any seven residues ending in either an Alanine (A) or an Asparagine (N) residue (FXXXXXXXY, wherein X is any amino acid and Y is A or N (SEQ ID NO: 2).

SEQ ID NO: 3 is the amino acid sequence of the human TR-3 peptide. The Lewis rat Class II MHC binding motif is QGGTRSPRCDCAG and comprises motif residues amino acid T and D and five intervening amino acids. Amino acids 14-32 represent an extended peptide which is shown to be the B cell epitope.

SEQ ID NO: 4 is an isolated nucleic acid sequence encoding TR-3 peptide.

SEQ ID NO: 5 is an altered TR3 peptide 1-32 to introduce a new MHC Class II binding motif (QGGFRSPRCDCAGDFHKKIGLFCCRGCPAGHY) that renders the altered TR3 peptide immunogenic for B cells in C57B1/6J mice.

SEQ ID NO: 6 is an altered (FITC) conjugated peptide containing a Lewis rat Class II binding motif (FITC-βAGGGITYLGKAGVAIGFSGTAPVDATG) at the carboxyl terminus.

### BRIEF DESCRIPTION OF THE DRAWINGS

FIG. 1 shows that Lewis rats primed with TR3 peptide 1-32 demonstrate an increased anti-TR3 peptide titer in their sera. Rats were immunized and rested for four weeks. Immune sera were assayed at 1:5000 and compared to pre-immune titers by fluorescence ELISA (see below) using TR3 peptide 1-32 coated plates.

FIG. 2 shows that Lewis rats primed with TR3 peptide 1-13 do not show an increase in antibody titer either against TR3 peptide 1-13 or TR3 peptide 1-32. Lewis rats were immunized with TR3 peptide 1-13 and after six weeks the animals were bled and sera tested for binding to TR31-13 or TR31-32 coated plates using fluorescence ELISA. No detectable antibody to either of the TR3 peptides was observed.

FIG. 3 shows that peptide 1-13 immune animals have a good T-cell response to the immunizing peptide. Lymph node T cells from TR3 peptide 1-13 immune animals were cultured in the presence of antigen presenting cells and either TR3 peptide 1-13 or TR3 peptide 14-32. ³H-thymidine incorporation was assessed after 48 hours in culture for an 18 hour period. The T cells responded in this standard T cell proliferation assay to TR3 peptide 1-13 but not to TR3 peptide 14-32. These data indicate that the TR3 peptide 1-13 peptide is immunogenic for T cells even though B cell responses are lacking. This implies that the missing component is a B cell epitope.

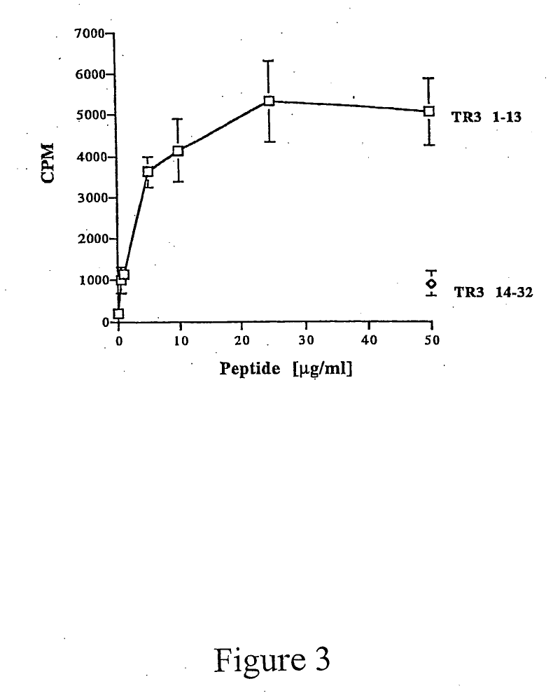

FIG. 4, shows that hybridomas from TR3 peptide 1-32 primed Lewis rats demonstrate specificity for the TR3 peptide 14-32 sequence and little affinity for TR3 peptide 1-13. Three different monoclonal antibodies (MAb) isolated from hybridomas derived from rats immunized with TR3 peptide 1-32 recognize TR3 peptide 14-32 and not TR3 peptide 1-13 as demonstrated in this fluorescence ELISA assay.

FIG. 5, shows that Altering TR3 peptide 1-32 to introduce a new MHC Class II binding motif (QGGFRSPRCDCAGDFHKKIGLFCCRGCPAGHY-SEQ ID NO: 5) renders the altered TR3 peptide immunogenic for B cells in C57B1/6J mice. C57B1/6J mice were immunized with the native TR3 peptide 1-32 or with an altered TR3 sequence that contains a Threonine (T) to Phenylalanine (F) alteration at amino acid position 4 to introduce a known C57B1/6J MHC Class II binding motif. Immune sera were collected after four weeks and assessed for anti-TR3 binding antibodies by fluorescence ELISA. None of the eight mice immunized with the native peptide had any detectable antibodies. In contrast, the four animals immunized with the T to F mutated peptide made an anti-TR3 response. This indicates that insertion of Class II binding motif is sufficient to induce an antibody in a previously non-immunogenic peptide.

FIG. 6, shows that Five Lewis rats were immunized with a fluorescein isothiocyanate (FITC) conjugated peptide containing a Lewis rat Class II binding motif (FITC-βAGGGITYLGKAGVAIGFSGTAPVDATG-SEQ ID NO: 6) at the carboxyl terminus. FITC was attached to the amino terminus of the growing peptide on the synthesizer by standard chemical means. The peptide was then released from the solid support according to standard methods. Sera were obtained from pre-immune animals and compared with sera obtained three weeks after priming. Anti-FITC antibodies were detected in a fluorescence ELISA using FITC conjugated bovine serum albumin coated plates. All five animals had an increase in anti-FITC antibodies. These data indicate that the MHC Class II binding motif can be positioned at the carboxyl terminus and still present a B cell epitope efficiently. These data further demonstrate the utility of making haptenic moieties immunogenic by coupling them to a Class II binding motif-containing sequence.

### MODES FOR CARRYING OUT THE INVENTION

General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual,” second edition (Sambrook et al., 1989); “Oligonucleotide Synthesis” (M. J. Gait, ed., 1984); “Animal Cell Culture” (R. I. Freshney, ed., 1987); the series “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987, and periodic updates); “PCR: The Polymerase Chain Reaction,” (Mullis et al., eds., 1994); “Current Protocols in Immunology” (J. E. Coligan et al., eds., 1991). All referenced books, patents, published applications and articles are incorporated by reference into the present disclosure to more fully describe the state of the art to which this invention pertains.

Definitions

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “native” or “natural” antigen is a polypeptide, protein or a fragment which contains an epitope, which has been isolated from a natural biological source, and which can specifically bind to an antigen receptor, in particular a T cell antigen receptor (TCR), in a subject.

The term “peptide” is used in its broadest sense to refer to a compound of two or more subunit amino acids, amino acid analogs, or peptidomimetics. The subunits may be linked by peptide bonds. In another embodiment, the subunit may be linked by other bonds, e.g. ester, ether, etc. As used herein the term “amino acid” refers to either natural and/or unnatural or synthetic amino acids, including glycine, beta amino acids and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein. Throughout this specification, numbering of amino acids in a peptide or polypeptide is from amino terminus to carboxy terminus.

The term “sequence motif” refers to a pattern present in a group of molecules.

The term “antigen” is well understood in the art as molecular species capable of inducing an immune response and of being recognized by antibody and/or sensitized cells manufactured as a consequence of the immune response.

The term “polynucleotide” and “nucleic acid molecule” are used interchangeably to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes, for example, single-, double-stranded and triple helical molecules, a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A nucleic acid molecule may also comprise modified nucleic acid molecules.

As used herein, “expression” refers to the process by which polynucleotides are transcribed into mRNA and translated into peptides, polypeptides, or proteins. If the polynucleotide is derived from genomic DNA, expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook, et al. (1989) Supra). Similarly, an eukaryotic expression vector includes a heterologous or homologous promoter for RNA polymerase II, a downstream polyadenylation signal, the start condon AUG, and a termination codon for detachment of the ribosome. Such vectors can be obtained commercially or assembled by the sequences described in methods well known in the art, for example, the methods described below for constructing vectors in general.

“Under transcriptional control” is a term well understood in the art and indicates that transcription of a polynucleotide sequence, usually a DNA sequence, depends on its being operably (operatively) linked to an element which contributes to the initiation of, or promotes, transcription. “Operably linked” refers to a juxtaposition wherein the elements are in an arrangement allowing them to function.

A “gene delivery vehicle” is defined as any molecule that can carry inserted polynucleotides into a host cell. Examples of gene delivery vehicles are liposomes, biocompatible polymers, including natural polymers and synthetic polymers; lipoproteins; polypeptides; polysaccharides; lipopolysaccharides; artificial viral envelopes; metal particles; and bacteria, viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include several non-viral vectors, including DNA/liposome complexes, and targeted viral protein-DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens, e.g., TCR, CD3 or CD4.

The term “major histocompatibility complex” or “MHC” refers to a complex of genes encoding cell-surface molecules that are required for antigen presentation to T cells and for rapid graft rejection. In humans, the MHC complex is also known as the HLA complex. The proteins encoded by the MHC complex are known as “MHC molecules” and are classified into class I and Class II MHC molecules. Class I MHC molecules include membrane heterodimeric proteins made up of an □ chain encoded in the MHC associated noncovalently with □2-microglobulin. Class I MHC molecules are expressed by nearly all nucleated cells and have been shown to function in antigen presentation to CD8⁺ T cells. Class II MHC molecules also include membrane heterodimeric proteins consisting of noncovalently associated alpha and beta chains. Class II MHC are known to participate in antigen presentation to CD4⁺ T cells and, in humans, include HLA-DP, -DQ, and DR. The term “MHC restriction” refers to a characteristic of T cells that permits them to recognize antigen only after it is processed and the resulting antigenic peptides are displayed in association with either a self class I or a self Class II MHC molecule. Methods of identifying and comparing MHC are well known in the art and are described in Allen et al. (1994) Human Imm. 40:25-32; Santamaria et al. (1993) Human Imm. 37:39-50 and Hurley et al. (1997) Tissue Antigens 50:401-415.

“Co-stimulatory molecules or receptors” are involved in the interaction between receptor-ligand pairs expressed on the surface of antigen presenting cells and T cells. Co-stimulatory molecules mediate co-stimulatory signal(s) which are necessary, under normal physiological conditions, to achieve full activation of naïve T cells. One exemplary receptor-ligand pair is the B7 co-stimulatory molecule on the surface of APCs and its counter-receptor CD28 or CTLA-4 on T cells (Freeman et al. (1993) Science 262:909-911; Young et al. (1992) J. Clin. Invest. 90: 229; Nabavi et al. (1992) Nature 360:266-268). Other important co-stimulatory molecules are CD40, CD54, CD80, CD86. The term “co-stimulatory molecule” encompasses any single molecule or combination of molecules which, when acting together with a peptide/MHC complex bound by a TCR on the surface of a T cell, provides a co-stimulatory effect which achieves activation of the T cell that binds the peptide. The term thus encompasses B7, or other co-stimulatory molecule(s) on an antigen-presenting matrix such as an APC, fragments thereof (alone, complexed with another molecule(s), or as part of a fusion protein) which, together with peptide/MHC complex, binds to a cognate ligand and results in activation of the T cell when the TCR on the surface of the T cell specifically binds the peptide. Co-stimulatory molecules are commercially available from a variety of sources, including, for example, Beckman Coulter. It is intended, although not always explicitly stated, that molecules having similar biological activity as wild-type or purified co-stimulatory molecules (e.g., recombinantly produced or variants thereof) are intended to be used within the spirit and scope of the invention.

As used herein, “solid phase support” or “solid support,” used interchangeably, is not limited to a specific type of support. Rather a large number of supports are available and are known to one of ordinary skill in the art. Solid phase supports include silica gels, resins, derivatized plastic films, glass beads, cotton, plastic beads, alumina gels. As used herein, “solid support” also includes synthetic antigen-presenting matrices, cells, and liposomes. A suitable solid phase support may be selected on the basis of desired end use and suitability for various protocols. For example, for peptide synthesis, solid phase support may refer to resins such as polystyrene (e.g., PAM-resin obtained from Bachem Inc., Peninsula Laboratories, etc.), POLYHIPE® resin (obtained from Aminotech, Canada), polyamide resin (obtained from Peninsula Laboratories), polystyrene resin grafted with polyethylene glycol (TentaGel®, Rapp Polymere, Tubingen, Germany) or polydimethylacrylamide resin (obtained from Milligen/Biosearch, California).

As used herein, the term “inducing an immune response in a subject” is a term well understood in the art and intends that an increase above background (from pre-immune titers) in an immune response to an antigen (or epitope) that can be detected (measured), after introducing the antigen (or epitope) into the subject, relative to the immune response (if any) before introduction of the antigen (or epitope) into the subject. An immune response to an antigen (or epitope), includes, but is not limited to, production of an antigen-specific (or epitope-specific) antibody, and production of an immune cell expressing on its surface a molecule which specifically binds to an antigen (or epitope). Methods of determining whether an immune response to a given antigen (or epitope) has been induced are well known in the art. For example, antigen-specific antibody can be detected using any of a variety of immunoassays known in the art, including, but not limited to, ELISA, wherein, for example, binding of an antibody in a sample to an immobilized antigen (or epitope) is detected with a detectably-labeled second antibody (e.g., enzyme-labeled mouse anti-human Ig antibody). Immune effector cells specific for the antigen can be detected any of a variety of assays known to those skilled in the art, including, but not limited to, FACS, or, in the case of CTLs, ⁵¹Cr-release assays, or in the case of T helper cells, ³H-thymidine uptake assays.

As used herein, the term “cytokine” refers to any one of the numerous factors that exert a variety of effects on cells, for example, inducing growth or proliferation. Non-limiting examples of cytokines which may be used alone or in combination in the practice of the present invention include, interleukin-2 (IL-2), stem cell factor (SCF), interleukin 3 (IL-3), interleukin 6 (IL-6), interleukin 12 (IL-12), G-CSF, granulocyte macrophage-colony stimulating factor (GM-CSF), interleukin-1 alpha (IL-1α), interleukin-11 (IL-11), MIP-1α, leukemia inhibitory factor (LIF), c-kit ligand, thrombopoietin (TPO) and flt3 ligand. Cytokines are commercially available from several vendors such as, for example, Genentech (South San Francisco, Calif.), Amgen (Thousand Oaks, Calif.), R&D Systems and Immunex (Seattle, Wash.). It is intended, although not always explicitly stated, that molecules having similar biological activity as wild-type or purified cytokines (e.g., recombinantly produced or variants thereof) are intended to be used within the spirit and scope of the invention.

A “subject” is a vertebrate, preferably a mammal, more preferably a human. Mammals include, but are not limited to, murines, simians, humans, farm animals, sport animals, and pets.

“PCR primers” refer to primers used in “polymerase chain reaction” or “PCR,” a method for amplifying a DNA base sequence using a heat-stable polymerase such as Taq polymerase, and two oligonucleotide primers, one complementary to the (+)-strand at one end of the sequence to be amplified and the other complementary to the (−)-strand at the other end. Because the newly synthesized DNA strands can subsequently serve as additional templates for the same primer sequences, successive rounds of primer annealing, strand elongation, and dissociation produce exponential and highly specific amplification of the desired sequence. (See, e.g., PCR 2: A PRACTICAL APPROACH, Supra). PCR also can be used to detect the existence of the defined sequence in a DNA sample.

“Host cell” or “recipient cell” is intended to include any individual cell or cell culture which can be or have been recipients for vectors or the incorporation of exogenous nucleic acid molecules, polynucleotides and/or peptides (or polypeptides). It also is intended to include progeny of a single cell, and the progeny may not necessarily be completely identical (in morphology or in genomic or total DNA complement) to the original parent cell due to natural, accidental, or deliberate mutation. The cells may be prokaryotic or eukaryotic, and include but are not limited to bacterial cells, yeast cells, animal cells, and mammalian cells, e.g., murine, rat, simian or human.

An “antibody” is an immunoglobulin molecule capable of binding an antigen. As used herein, the term encompasses not only intact inmunoglobulin molecules, but also anti-idiotypic antibodies, mutants, fragments, fusion proteins, bi-specific antibodies, humanized proteins and modifications of the immunoglobulin molecule that comprise an antigen recognition site of the required specificity.

An “antibody complex” is the combination of antibody (as defined above) and its binding partner or ligand.

The term “isolated” means separated from constituents, cellular and otherwise, in which the polynucleotide, peptide, polypeptide, protein, antibody, or fragments thereof, are normally associated with in nature. For example, with respect to a polynucleotide, an isolated polynucleotide is one that is separated from the 5′ and 3′ sequences with which it is normally associated in the chromosome. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated,” “separated” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Although not explicitly stated for each of the inventions disclosed herein, it is to be understood that all of the above embodiments for each of the compositions disclosed below and under the appropriate conditions, are provided by this invention. Thus, a non-naturally occurring polynucleotide is provided as a separate embodiment from the isolated naturally occurring polynucleotide. A protein produced in a bacterial cell is provided as a separate embodiment from the naturally occurring protein isolated from a eukaryotic cell in which it is produced in nature.

An “isolated” population of cells is “substantially free” of cells and materials with which it is associated in nature. By “substantially free” or “substantially pure” means at least 50% of the population are the desired cell type, preferably at least 70%, more preferably at least 80%, and even more preferably at least 90%.

A “composition” is intended to mean a combination of active agent and another compound or composition, inert (for example, a detectable agent, solid support or label) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON's PHARM. SCI., 15th Ed. (Mack Publ. Co., Easton (1975)).

An “effective amount” is an amount sufficient to effect beneficial or desired results. An effective amount can be administered in one or more administrations, applications or dosages. In the context of a disease state, an effective amount of an immunomodulatory agent of the invention, including a peptide of the invention or a polynucleotide of the invention is an amount that is sufficient to palliate, ameliorate, stabilize, reverse, slow or delay the progression of the disease state.

A synthetic immunogen is provided by this invention. The immunogen contains a first peptide component that functions as a B cell epitope, linked to a second peptide component that comprises at least one MHC Class II binding motif and a T cell epitope. The synthetic peptides can be made by 1) the synthesis of native amino acid sequence long enough to contain both components by increasing the length of the peptide beyond the usual T cell epitope length of 8-15 residues to ones extended in length to contain a B cell epitope; 2) the synthesis of a sequence containing a known B cell epitope adjacent to non-native amino acid sequences containing a known Class II binding motif; 3) the synthesis of elongated native peptide sequence mutated to contain a known Class II binding; or 4) the synthesis of a peptide sequence containing a containing a known Class II binding motif as described above (1-3) but then chemically modified to contain B cell determinants such as a carbohydrate, lipid or other chemical structures. In other words, the immunogen can be an assembly of separate components isolated form non-contiguous amino acid sequences. Methods for linking peptide components are known in the art, and include, but are not limited to chemical conjugation and a peptide bond. Alternatively, a contiguous amino acid sequence can be modified by example, deletion of non-essential amino acids and/or substitution to create at least one MHC class T binding motif.

The first peptide component comprising a B cell epitope can be isolated, for example, from a carbohydrate, a lipid, or a peptide. Examples of suitable peptides, include, but are not limited to a bacterial peptide, a viral peptide, a parasitic peptide, a plant peptide, a fungal peptide, a reptilian peptide, an arachnid peptide, a human peptide, a bovine peptide, a feline peptide, a canine peptide, an equine peptide, a porcine peptide, a murine peptide or a rattus peptide.

Alternatively, the first peptide component comprises a native peptide isolated from a T cell inhibitory protein. In another aspect, the first peptide component comprises a synthetic peptide synthesized from a native T cell inhibitory protein. Examples of T cell inhibitory proteins are provided infra.

It is expected that not all peptide sequences containing a Class II binding motif will give rise to antibodies that recognize the native three dimentional structure of a protein. Although antibodies to the linear sequence of amino acids is of value for the analysis of protein expression, those to protective B cell epitopes will require recognition of the native conformation of the protein. In order to assure that antibodies will be directed against external or exposed portions of proteins Class II binding motifs could be placed adjacent to peptide segments of the proteins predicted to be exposed based upon computer programs that predict folding patterns of proteins, for example, the homology modeling package, FOLDER (Srinivasan et al, Protein Science 2:277-289, 1993). The modeling program called SYBYL (Tripos, Inc.) is another suitable example.

Some simple monosaccharides are known to be B cell epitopes. Rhamnose for example has been identified as the dominant protective determinant for group B Streptococci. Rhamnopyranosylphenyl isothiocyanate is available (SIGMA-Aldrich) and can be readily conjugated to the amino terminus of a peptide as described in sequence ID No. 6 for fluorescein isothiocyanate.

The synthetic immunogens or peptide components used in accordance with the method of the present invention can be obtained in any one of a number of conventional ways. For example, they can be prepared by chemical synthesis using standard techniques such as solid phase peptide synthesis techniques. Automated peptide synthesizers are commercially available, as are the reagents required for their use. See, e.g., Steward and Young, eds. (1968) SOLID PHASE PEPTIDE SYNTHESIS, Freemantle, San Francisco, Calif. A preferred method is the Merrifield process. Merrifield (1967) Recent progress in Hormone Res. 23:451.

Alternatively, the peptides can be prepared by enzymatic digestion or cleavage of naturally occurring proteins. The peptides can also be prepared using recombinant techniques known to those of skill in the art.

Once an isolated peptide of the invention is obtained, it may be purified by standard methods including chromatography (e.g., ion exchange, affinity, and sizing column chromatography), centrifugation, differential solubility, or by any other standard technique for protein purification. For immunoaffinity chromatography, an epitope may be isolated by binding it to an affinity column comprising antibodies that were raised against that peptide, or a related peptide and were affixed to a stationary support.

Alternatively, affinity tags such as hexa-His (Invitrogen), Maltose binding domain (New England Biolabs), influenza coat sequence (Kolodziej et al. (1991) Methods Enzymol. 194:508-509), and glutathione-S-transferase can be attached to the peptides allow easy purification by passage over an appropriate affinity column. Isolated peptides can also be physically characterized using such techniques as proteolysis, nuclear magnetic resonance, and x-ray crystallography.

A synthetic immunogen of the invention can be used in a variety of formulations, which may vary depending on the intended use. For example, a peptide of the invention can be covalently or non-covalently complexed to a macromolecular carrier, including, but not limited to, natural and synthetic polymers, proteins, polysaccharides, poly(amino acid), polyvinyl alcohol, polyvinyl pyrrolidone, and lipids. A peptide can be conjugated to a fatty acid, for introduction into a liposome. U.S. Pat. No. 5,837,249. A synthetic peptide of the invention can be complexed covalently or non-covalently with a solid support, a variety of which are known in the art. Examples of protein carriers include, but are not limited to, superantigens, serum albumin, tetanus toxoid, ovalbumin, thyroglobulin, myoglobulin, and immunoglobulin.

The peptides of this invention also can be combined with various liquid phase carriers, such as sterile or aqueous solutions, pharmaceutically acceptable carriers, suspensions and emulsions. Examples of non-aqueous solvents include propyl ethylene glycol, polyethylene glycol and vegetable oils. When used to prepare antibodies, the carriers also can include an adjuvant that is useful to non-specifically augment a specific immune response. A skilled artisan can easily determine whether an adjuvant is required and select one. However, for the purpose of illustration only, suitable adjuvants include, but are not limited to, Freund's Complete and Incomplete, mineral salts and polynucleotides.

The synthetic immunogens of this invention are useful in vitro in antibody-binding assays or alternatively, coupled to solid support for use in purifying antibodies from solution. For example, detectably labeled peptides and polypeptides can be bound to a column and used for the detection and purification of antibodies. Also provided by this application are the synthetic immunogens conjugated to a detectable agent for use in the diagnostic methods.

They can be used in vito to raise immune response as treatment for a variety of disease, e.g., to treat a bacterial infection, or alternatively, to prevent infection or disease (prophylacticly) as in a vaccine composition.

The compositions of this invention can optionally contain an effective amount of a cytokine and/or a co-stimulatory molecule.

When used in vivo pharmaceutically, an effective amount of the peptide is delivered or administered to the subject or animal to be treated. In one aspect, the immunogen is delivered as a vaccine and can optionally contain an effective amount of an adjuvant.

Also provided by this invention are isolated nucleic acids or polynucleotides encoding the immunogens of this invention. In one aspect, the polynucleotide comprises, in a 5′ to 3′ orientation, one or more polynucleotides encoding transcription and translation elements and the elements of the immunogen: a codon for an amino acid identified as a Class II binding motif element, followed by codons for a sequence of amino acids for the proper spacing between motif elements, next a codon for the second Class II binding motif element followed by the sequence encoding the antigenic element of sufficient length to lie outside a Class II binding site. In a further aspect, a polynucleotide encoding the immunogen is optionally inserted between transcription enhancer or promotor elements.

The polynucleotides can be conjugated to a detectable marker, e.g., an enzymatic label or a radioisotope for detection of nucleic acid and/or expression of the gene in a cell. A wide variety of appropriate detectable markers are known in the art, including fluorescent, radioactive, enzymatic or other ligands, such as avidin/biotin, which are capable of giving a detectable signal. One may likely desire to employ a fluorescent label or an enzyme tag, such as urease, alkaline phosphatase or peroxidase, instead of radioactive or other environmental undesirable reagents. In the case of enzyme tags, colorimetric indicator substrates are known which can be employed to provide a means visible to the human eye or spectrophotometrically, to identify specific hybridization with complementary nucleic acid-containing samples. Briefly, this invention further provides a method for detecting a single-stranded polynucleotide or its complement, by contacting target single-stranded polynucleotides with a labeled, single-stranded polynucleotide (a probe) which is at least 4, and more preferably at least 5 or 6 and most preferably at least 10 of the 10 nucleotides of a polynucleotide of the invention (or the corresponding complement) under conditions permitting hybridization (preferably moderately stringent hybridization conditions) of complementary single-stranded polynucleotides, or more preferably, under highly stringent hybridization conditions. Hybridized polynucleotide pairs are separated from un-hybridized, single-stranded polynucleotides. The hybridized polynucleotide pairs are detected using methods well known to those of skill in the art and set forth, for example, in Sambrook et al. (1989) supra.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions of about 6×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The polynucleotides of this invention can be replicated using PCR. PCR technology is the subject matter of U.S. Pat. Nos. 4,683,195; 4,800,159; 4,754,065; and 4,683,202 and described in PCR: THE POLZMERASE CHAIN REACTION (Mullis et al. eds., Birkhauser Press, Boston (1994)) and references cited therein.

Alternatively, one of skill in the art can use the sequences provided herein and a commercial DNA synthesizer to replicate the DNA. Accordingly, this invention also provides a process for obtaining the polynucleotides of this invention by providing the linear sequence of the polynucleotide, appropriate primer molecules, chemicals such as enzymes and instructions for their replication and chemically replicating or linking the nucleotides in the proper orientation to obtain the polynucleotides. In a separate embodiment, these polynucleotides are further isolated. Still further, one of skill in the art can insert the polynucleotide into a suitable replication vector and insert the vector into a suitable host cell (prokaryotic or eukaryotic) for replication and amplification. The DNA so amplified can be isolated from the cell by methods well known to those of skill in the art. A process for obtaining polynucleotides by this method is further provided herein as well as the polynucleotides so obtained.

RNA can be obtained by first inserting a DNA polynucleotide into a suitable host cell. The DNA can be inserted by any appropriate method, e.g., by the use of an appropriate gene delivery vehicle (e.g., liposome, plasmid or vector) or by electroporation. When the cell replicates and the DNA is transcribed into RNA; the RNA can then be isolated using methods well known to those of skill in the art, for example, as set forth in Sambrook et al. (1989) supra. For instance, mRNA can be isolated using various lytic enzymes or chemical solutions according to the procedures set forth in Sambrook et al. (1989) supra or extracted by nucleic-acid-binding resins following the accompanying instructions provided by manufactures.

The invention further provides the isolated polynucleotide operatively linked to a promoter of RNA transcription, as well as other regulatory sequences for replication and/or transient or stable expression of the DNA or RNA. As used herein, the term “operatively linked” means positioned in such a manner that the promoter will direct transcription of RNA off the DNA molecule. Examples of such promoters are SP6, T4 and T7. In certain embodiments, cell-specific promoters are used for cell-specific expression of the inserted polynucleotide. Vectors which contain a promoter or a promoter/enhancer, with termination codons and selectable marker sequences, as well as a cloning site into which an inserted piece of DNA can be operatively linked to that promoter are well known in the art and commercially available. For general methodology and cloning strategies, see GENE EXPRESSION TECHNOLOGY (Goeddel ed., Academic Press, Inc. (1991)) and references cited therein and VECTORS: ESSENTIAL DATA SERIES (Gacesa and Ramji, eds., John Wiley & Sons, N.Y. (1994)), which contains maps, functional properties, commercial suppliers and a reference to GenEMBL accession numbers for various suitable vectors. Preferably, these vectors are capable of transcribing RNA in vitro or in vivo.

The present invention also provides delivery vehicles suitable for delivery of a polynucleotide of the invention into cells (whether in vivo, ex vivo, or in vitro). A polynucleotide of the invention can be contained within a cloning or expression vector. These vectors (especially expression vectors) can in turn be manipulated to assume any of a number of forms which may, for example, facilitate delivery to and/or entry into a cell.

Expression vectors containing these nucleic acids are useful to obtain host vector systems to produce proteins and polypeptides. It is implied that these expression vectors must be replicable in the host organisms either as episomes or as an integral part of the chromosomal DNA. Suitable expression vectors include plasmids, viral vectors, including adenoviruses, adeno-associated viruses, retroviruses, cosmids, etc. Adenoviral vectors are particularly useful for introducing genes into tissues in vivo because of their high levels of expression and efficient transformation of cells both in vitro and in vivo. When a nucleic acid is inserted into a suitable host cell, e.g., a prokaryotic or a eukaryotic cell and the host cell replicates, the protein can be recombinantly produced. Suitable host cells will depend on the vector and can include mammalian cells, animal cells, human cells, simian cells, insect cells, yeast cells, and bacterial cells constructed using well known methods. See Sambrook, et al. (1989) supra. In addition to the use of viral vector for insertion of exogenous nucleic acid into cells, the nucleic acid can be inserted into the host cell by methods well known in the art such as transformation for bacterial cells; transfection using calcium phosphate precipitation for mammalian cells; or DEAE-dextran; electroporation; or microinjection. See Sambrook et al. (1989) supra for this methodology. Thus, this invention also provides a host cell, e.g., a mammalian cell, an animal cell (rat or mouse), a human cell, or a prokaryotic cell such as a bacterial cell, containing a polynucleotide encoding a protein or polypeptide or antibody.

When the vectors are used for gene therapy in vivo or ex vivo, a pharmaceutically acceptable vector is preferred, such as a replication-incompetent retroviral or adenoviral vector. Pharmaceutically acceptable vectors containing the nucleic acids of this invention can be further modified for transient or stable expression of the inserted polynucleotide. As used herein, the term “pharmaceutically acceptable vector” includes, but is not limited to, a vector or delivery vehicle having the ability to selectively target and introduce the nucleic acid into dividing cells. An example of such a vector is a “replication-incompetent” vector defined by its inability to produce viral proteins, precluding spread of the vector in the infected host cell. An example of a replication-incompetent retroviral vector is LNL6. Miller et al. (1989) BioTechniques 7:980-990. The methodology of using replication-incompetent retroviruses for retroviral-mediated gene transfer of gene markers is well established. Correll et al. (1989) Proc. Natl. Acad. Sci. USA 86:8912; Bordignon (1989) Proc. Natl. Acad. Sci. USA 86:8912-52; Culver (1991) Proc. Natl. Acad. Sci. USA 88:3155; and Rill (1991) Blood 79(10):2694-700.

In general, genetic modifications of cells employed in the present invention are accomplished by introducing a vector containing a polynucleotide comprising sequences encoding a synthetic immunogen of the invention. A variety of different gene transfer vectors, including viral as well as non-viral systems can be used.

A wide variety of non-viral vehicles for delivery of a polynucleotide of the invention are known in the art and are encompassed in the present invention. A polynucleotide of the invention can be delivered to a cell as naked DNA. WO 97/40163. Alternatively, a polynucleotide of the invention can be delivered to a cell associated in a variety of ways with a variety of substances (forms of delivery) including, but not limited to cationic lipids; biocompatible polymers, including natural polymers and synthetic polymers; lipoproteins; polypeptides; polysaccharides; lipopolysaccharides; artificial viral envelopes; metal particles; and bacteria. A delivery vehicle may take the form of a microparticle. Mixtures or conjugates of these various substances can also be used as delivery vehicles. A polynucleotide of the invention can be associated with these various forms of delivery non-covalently or covalently.

Included in the non-viral vector category are prokaryotic plasmids and eukaryotic plasmids. Non-viral vectors (i.e., cloning and expression vectors) having cloned therein a polynucleotide(s) of the invention can be used for expression of recombinant polypeptides as well as a source of polynucleotide of the invention. Cloning vectors can be used to obtain replicate copies of the polynucleotides they contain, or as a means of storing the polynucleotides in a depository for future recovery. Expression vectors (and host cells containing these expression vectors) can be used to obtain polypeptides produced from the polynucleotides they contain. They may also be used where it is desirable to express polypeptides, encoded by an operably linked polynucleotide, in an individual, such as for eliciting an immune response via the polypeptide(s) encoded in the expression vector(s). Suitable cloning and expression vectors include any known in the art, e.g., those for use in bacterial, mammalian, yeast and insect expression systems. Specific vectors and suitable host cells are known in the art and need not be described in detail herein. For example, see Gacesa and Ramji, Vectors, John Wiley & Sons (1994).

Cloning and expression vectors typically contain a selectable marker (for example, a gene encoding a protein necessary for the survival or growth of a host cell transformed with the vector), although such a marker gene can be carried on another polynucleotide sequence co-introduced into the host cell. Only those host cells into which a selectable gene has been introduced will survive and/or grow under selective conditions. Typical selection genes encode protein(s) that (a) confer resistance to antibiotics or other toxins substances, e.g., ampicillin, neomycyin, methotrexate, etc.; (b) complement auxotrophic deficiencies; or (c) supply critical nutrients not available from complex media. The choice of the proper marker gene will depend on the host cell, and appropriate genes for different hosts are known in the art. Cloning and expression vectors also typically contain a replication system recognized by the host.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, Co1E1, pCR1, RP4, phage DNAs, and shuttle vectors such as pSA3 and pAT28. These and many other cloning vectors are available from commercial vendors such as BioRad, Strategene, and Invitrogen. The Examples provided herein also provide examples of cloning vectors.

Expression vectors generally are replicable polynucleotide constructs that contain a polynucleotide encoding a polypeptide of interest. The polynucleotide encoding the polypeptide of interest is operably linked to suitable transcriptional controlling elements, such as promoters, enhancers and terminators. For expression (i.e., translation), one or more translational controlling elements are also usually required, such as ribosome binding sites, translation initiation sites, and stop codons. A polynucleotide sequence encoding a signal peptide can also be included to allow a polypeptide, encoded by an operably linked polynucleotide, to cross and/or lodge in cell membranes or be secreted from the cell. A number of expression vectors suitable for expression in eukaryotic cells including yeast, avian, and mammalian cells are known in the art. Examples of mammalian expression vectors contain both prokaryotic sequence to facilitate the propagation of the vector in bacteria, and one or more eukaryotic transcription units that are expressed in eukaryotic cells. Examples of mammalian expression vectors suitable for transfection of eukaryotic cells include the pcDNAI/amp, pcDNAI/neo, pRc/CMV, pSV2gpt, pSV2neo, pRSVneo, and pHyg derived vectors. Alternatively, derivatives of viruses such as the bovine papilloma virus (BPV-1), or Epstein-Barr virus (pHEB, pREP derived vectors) can be used for expression in mammalian cells. Examples of expression vectors for yeast systems, include YEP24, YIP5, YEP51, YEP52, YES2 and YRP17, which are cloning and expression vehicles useful for introduction of constructs into S. cerevisiae. Broach et al. (1983) experimental manipulation of gene expression, ed. M. Inouye, Academic Press. p. 83. Baculovirus expression vectors for expression in insect cells include pVL-derived vectors (such as pVL1392, pVL1393 and pVL941), pAcUW-derived vectors and pBlueBac-derived vectors.

Viral vectors include, but are not limited to, DNA viral vectors such as those based on adenoviruses, herpes simplex virus, poxviruses such as vaccinia virus, and parvoviruses, including adeno-associated virus; and RNA viral vectors, including, but not limited to, the retroviral vectors. Retroviral vectors include murine leukemia virus, and lentiviruses such as human immunodeficiency virus. Naldini et al. (1996) Science 272:263-267.

Replication-defective retroviral vectors harboring a polynucleotide of the invention as part of the retroviral genome can be used. Such vectors have been described in detail. (Miller et al. (1990) Mol. Cell Biol. 10:4239; Kolberg, R. (1992) J. NIH Res. 4:43; Cornetta et al. (1991) Hum. Gene Ther 2:215).

Adenovirus and adeno-associated virus vectors useful in the genetic modifications of this invention may be produced according to methods already taught in the art. (See, e.g., Karlsson et al. (1986) EMBO 5:2377; Carter (1992) Current Opinion in Biotechnology 3:533-539; Muzcyzka (1992) Current Top. Microbiol. Immunol. 158:97-129; GENE TARGETING: A PRACTICAL APPROACH (1992) ed. A. L. Joyner, Oxford University Press, NY). Several different approaches are feasible.

Additional references describing viral vectors which could be used in the methods of the present invention include the following: Horwitz, M. S., Adenovinidae and Their Replication, in Fields, B., et al. (eds.) VIROLOGY, Vol. 2, Raven Press New York, pp.1679-1721, 1990); Graham, F. et al., pp. 109-128 in METHODS IN MOLECULAR BIOLOGY, Vol.7: GENE TRANSFER AND EXPRESSION PROTOCOLS, Murray, E. (ed.), Humana Press, Clifton, N.J. (1991); Miller et al. (1995) FASEB Journal 9:190-199, Schreier (1994) Pharmaceutica Acta Helvetiae 68:145-159; Schneider and French (1993) Circulation 88:1937-1942; Curiel et al. (1992) Human Gene Therapy 3:147-154; Graham et al. WO 95/00655 (5 Jan. 1995); Falck-Pedersen WO 95/16772 (22 Jun. 1995); Denefle et al. WO 95/23867 (8 Sep. 1995); Haddada et al. WO 94/26914 (24 Nov. 1994); Perricaudet et al. WO 95/02697 (26 Jan. 1995); and Zhang et al. WO 95/25071 (12 Oct. 1995).

Isolated host cells containing the synthetic immunogens of this invention and/or the polynucleotides encoding them are also provided by this invention. The host cell can be a prokaryotic cell or a eukaryotic cell. Examples of suitable prokaryotic host cells include, but are not limited to E. coli, Listeria monocytogenes and Salmonella sp. Examples of suitable eukaryotic host cells include but are not limited to autologous dendritic cells, macrophages and B cells.

Any of the immunogens, polynucleotides or host cells can be further combined with a cytokine or co-stimulatory molecule or a polynucleotide encoding each and/or both.

The compositions of this invention are useful to raise immune serum by delivering to an animal an effective amount of one or more of the synthetic immunogen, polynucleotide, host cell and pharmaceutical compositions containing them. The method can further comprise delivering an effective amount of a cytokine or co-stimulatory molecule to the animal in the form of a peptide(s) or polynucleotide(s) encoding the peptide(s). Examples of suitable co-stimulatory molecules include, but are not limited to CD28, CD80 (B7-2), CD86 (B7-1), CD134 (OX40), CDw137 (4-1BB), CD152 (CTLA-4), CD153 (CD30 ligand) and CD154 (CD40 ligand).

When delivered to an animal such as a mouse or rat, the method provides an animal model to test new therapies and/or compositions. In one aspect, the animal expresses a human immunoglobulin gene, examples of which include, but are not limited to IgA1, IgaA2, IgM, IgD, IgG1, IgG2, IgG3, and IgG4.

When delivered to a human patient, the method is useful to raise an immune response in the subject. As is known to those of skill in the art, the desired response, whether therapeutic, prophylatic or specific for a disease or condition, can be pre-selected by use of the appropriate B cell epitope. For example, the sugar rhamnose is identified as a protective epitope of Group B Streptococci.

Further provided by this invention is a method for producing a hybridoma cell line producing monoclonal antibody specific for a B cell epitope as well as the hybridoma cell line and monoclonal antibody produced by this method. The method requires fusing antibody producing cells isolated from an animal immunized with a composition of this invention containing the B cell epitope with at least one myeloma cell, thereby producing the hybridoma cell line specific for the B cell epitope of said peptide. Examples of antibody producing cells include but are not limited to those isolated from a lymph node, spleen or other lymphoid organ.

The monoclonal antibodies of the invention also can be bound to many different carriers. Thus, this invention also provides compositions containing the antibodies and another substance, active or inert. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding monoclonal antibodies, or will be able to ascertain such, using routine experimentation.

Compositions containing the antibodies, fragments thereof or cell lines which produce the antibodies, are encompassed by this invention. When these compositions are to be used pharmaceutically, they are combined with a pharmaceutically acceptable carrier.

A method of producing monoclonal antibody to a T cell inhibitory molecule also is provided by this invention. The method is performed by the steps of contacting in vitro, a lymphoid cell isolated from an animal immunized with a synthetic peptide, wherein the synthetic peptide comprises a T cell inhibitory molecule that contains a MHC Class II binding motif and a B cell epitope, with an effective amount of a composition comprising a T cell stimulation factor and with an effective amount of T cells that specifically recognize and bind the T cell inhibitory molecule that contains a MHC Class II binding motif to produce an antibody producing lymphoid cell. Then, the cell of step a) is fused with at least one myeloma cell, to produce a hybridoma cell line that produces a monoclonal antibody to a T cell inhibitory molecule.

The method can further comprise delivering an effective amount of a cytokine or co-stimulatory molecule to the animal in the form of a peptide(s) or polynucleotide(s) encoding the peptide(s). Examples of suitable co-stimulatory molecules include, but are not limited to CD28, CD80 (B7-2), CD86 (B7-1), CD134 (OX40), CDw137 (4-1BB), CD152 (CTLA-4), CD153 (CD30 ligand) and CD154 (CD40 ligand).

Human monoclonal antibodies can be produced by use of a humanized animal, e.g., humanized rat or mouse, in this method. In a further aspect, the animal from which the lymphoid cell has been isolated expresses MHC Class II gene that may be associated with an identifiable MHC Class II binding motif. In yet a further aspect, extended peptide was delivered to the animal alone or with an adjuvant.

In another aspect, the T cell inhibitory molecule comprises an extended peptide that is altered to create a MHC Class II binding motif where none previously existed or the native amino acid sequence of the extended peptide. Examples of such include, but are not limited to peptides selected from the group consisting of a death domain receptor, a growth hormone receptor, a growth hormone and a co-stimulatory receptor or biologically active fragments thereof having the same or similar biological activity as full length protein. In a further aspect, the T cell inhibitory molecule is selected from the group consisting of CD25, CD26, CD30, CD49a, CD69, CD70, CD83, CD87, CD96, CD97, CDw108, and CD109.

Examples of suitable death domain receptor peptides include, but are not limited to those selected from the group CD95 (Fas, Apo1), TR3 (DR3, Apo3, WSL-1, TRAMP, LARD), DR4, DR5 (Apo2, TRAIL-R2, TRICK, KILLER) DR6, TNFR1 and p55 (CD120a).

Examples of suitable growth hormone receptors include, but are not limited to those selected from the group consisting of: CD25 (IL-2 R alpha chain), CD119 (IFN gamma, alpha chain), CD121a (IL-1R, type I), CD121b (IL-1R, type II), CD122 (IL-2R, beta chain), CD123 (IL-3R, alpha chain), CD124 (IL-4R), CD126 (IL-6R, alpha chain), CD127 (IL-7R, alpha chain) and CD132 (common gamma chain IL-2R/4R/7R/9R/15R).

Examples of suitable growth hormones include, but are not limited to IL-1alpha, IL-1beta, IL-2, IL-3, IL-4, IL-5, IL-6, IL-7, IL-8, IL-9, IL-10, IL-12, IL-13, IL-14, IL-15, IL-16, IL-17 and IL-18.

The lymphoid cell and/or T cell can be isolated from a lymph node, spleen or other lymphoid organ.

The monoclonal antibodies of this invention can be used to induce or modulating an immune response to an antigen by delivering to an animal an effective amount of the monoclonal antibody.

The following examples are intended to illustrate, not limit the invention.

The amino acid sequence of the human TR3 gene product was searched for the presence of the Lewis rat Class II binding motif sequence. Four were found, two were on the extracellular domain and two on the cytoplasmic domain. One of the extracellular motifs was at the amino terminus at amino acid positions 4-10 (Table 1).

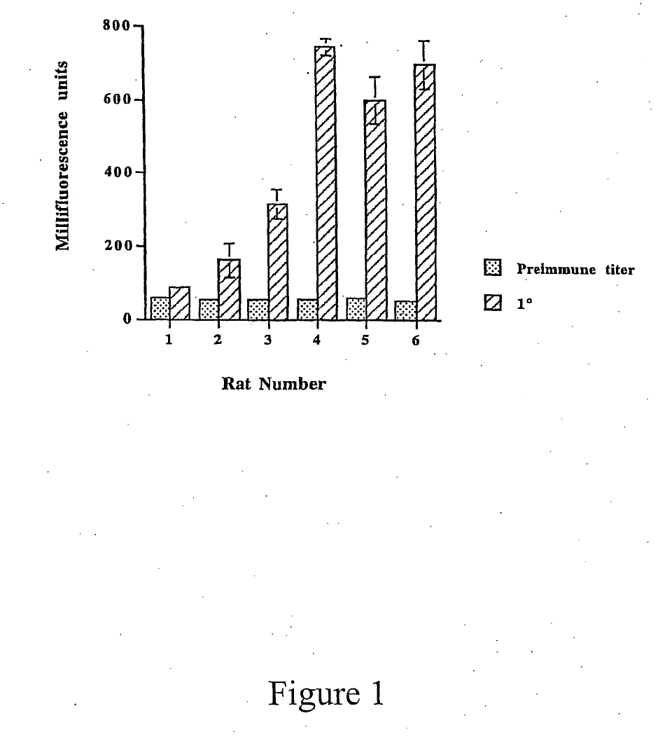

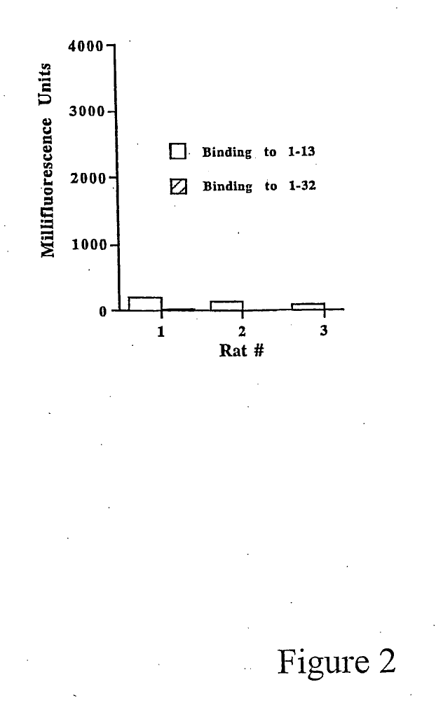

Two peptides were synthesized, TR3 peptide 1-13 and TR3 peptide 1-32. Both contain the binding motif but only one was long enough to have a portion hanging outside of the MHC Class II binding cleft. Lewis rats were immunized with one or the other of these peptides and sera tested for the presence of antibodies that would recognize the TR3₁₋₃₂ peptide before and after immunization. As shown in FIG. 1A, rats immunized with TR3 peptide had no appreciable antibody titer. In contrast, rats immunized with TR3 peptide 1-32 had a rigorous humoral immune response (FIG. 1B). The lack of an antibody response was not due to ineffective T cell help because the TR3 peptide 1-13 induced very strong T cell responses as demonstrated by the TR3 peptide 1-13 response in a standard proliferation assay (FIG. 2) of a T cell line derived from the draining lymph node as described previously (Wegmann et al., (1994) J. Immunol. 153:892-900).

The serum from rats immunized with TR3 peptide 1-32 were tested in a standard ELISA for epitope specificity. The protocol for ELISA has been described previously in (Tittle, Molecular Immunology 26:343-350, 1989), except that the substrate (methylumbelliferyl phosphate, MUP, Sigma-Aldrich, St. Louis, Mo.) was read fluorometrically rather than calorimetrically as with NPP (Nitrophenyl phosphate).

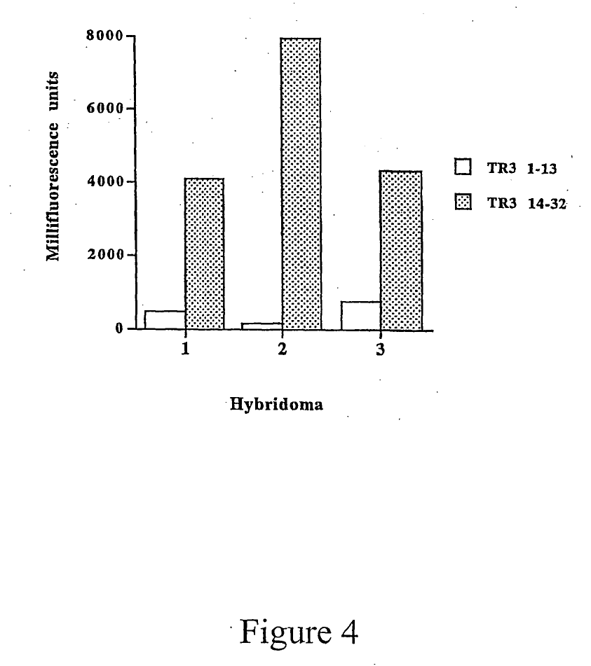

Sera from immune rats were tested for antibody binding to TR3 peptide 1-13 or TR3 peptide 14-32. The results shown in FIG. 4 demonstrate that the antibodies recognize TR3 peptide 14-32 but fail to recognize TR3 peptide 1-13. Thus, these findings are consistent with the de novo induction of both T cell and B cells to distinct epitopes by using an extended length peptide containing a Class II binding motif.

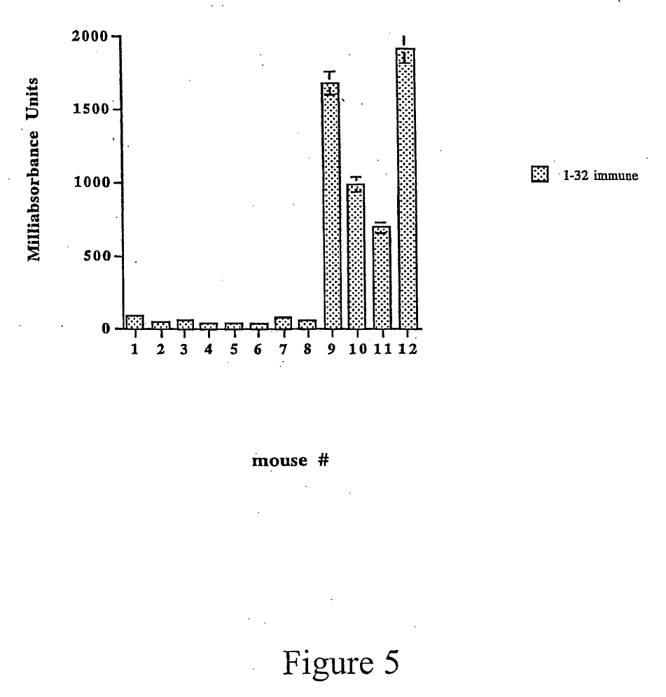

C57B1/6J mice that have a known peptide binding motif shown in Sequence No. 5 were used to show that non-immunogenic peptides can be altered to induce immune response. A T to F change in the sequence of TR3 at AA4 an H-2^{b} binding motif was generated and C57B1/6J mice were immunized with the native TR3 peptide or with the T-F variant peptide. The results of a typical experiment are shown in FIG. 5. The native TR31-32 peptide containing no known binding motif was not immunogenic in C57B1/6J mice. In contrast the nice immunized with the T-F variant peptide containing the motif was immunogenic and induced an antibody response. Thus, the ability to induce humoral antibody responses using an extended Class II bind motif was observed in two species and the motif can be inserted to generate immunogenic molecules where none existed previously.

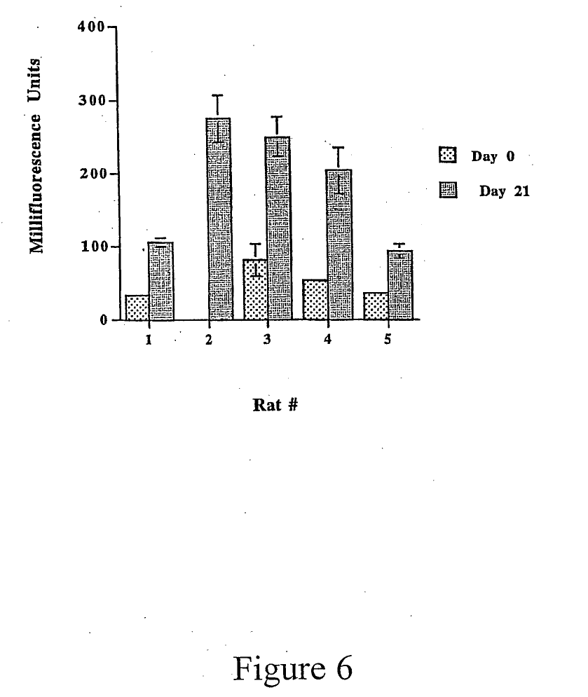

The ability of peptides with a Class II binding motif at the carboxyl terminus to induce B cell responses was tested using a pigeon cytochrome C peptide of known immunogenicity for C57B1/6J T cells (Itoh et al., Proc. Nat'l Acad Sci 94:12047, 1997). The peptide was altered to have the Class II binding motif at the carboxyl terminus. It was also changed to contain a Lewis Class II binding motif (See Sequence ID No. 6). Finally, the peptide was fluoresceinated during synthesis at the amino terminus. The ability of the monovalent fluoresceinated peptide to induce an anti-fluorescein response in Lewis rats was determined. Five Lewis rats were immunized with 200 □g of FITC:peptide in CFA and rested for three weeks. Preimmune serum titers were then compared with the day 21 immune serum titers. As shown in FIG. 6, all five animals had an increase in the anti-FITC antibody over their preimmune titers. These data demonstrate that the presentation of B cell epitopes can also be achieved by having the Class II binding motif at the carboxyl terminus. Further, these data demonstrate that monovalent conjugates are equally immunogenic. This opens up the door to the production of potent vaccines for future of mankind's needs.

Although the foregoing invention has been described in some detail by way of illustration and example for purposes of clarity of understanding, it will be apparent to those skilled in the art that certain changes and modifications will be practiced. Therefore, the description and examples should not be construed as limiting the scope of the invention, which is delineated by the appended claims.

1. An synthetic immunogen comprising a first peptide component comprising a B cell epitope linked to a second peptide component comprising at least one MHC Class II binding motif as a T cell epitope.

2. The immunogen of claim 1, wherein the first peptide component is linked to the second peptide component by chemical conjugation, a peptide bond, or an amino acid substitution to create a MHC Class II binding motif where none exists in the native amino acid sequence.

3. A composition comprising the immunogen of claim 1 and a substrate.

4. The composition of claim 3, wherein the substrate is alum.

5. The composition of claim 4, further comprising an effective amount of a cytokine and/or a co-stimulatory molecule.

6. The composition of claim 4, further comprising an adjuvant.

7. The composition of claim 1, wherein the first peptide component comprises a B cell epitope isolated from a component selected from the group consisting essentially of a carbohydrate, a lipid, and a peptide.

8. The composition of claim 7, wherein the peptide is selected from the group consisting essentially of a bacterial peptide, a viral peptide, a parasitic peptide, a plant peptide, a fungal peptide, a reptilian peptide, an arachnid peptide, a human peptide, a bovine peptide, a feline peptide, a canine peptide, an equine peptide, a porcine peptide, a murine peptide and a rattus peptide.

9. The composition of claim 1, wherein the first peptide component comprises a native peptide isolated from a T cell inhibitory protein.

10. The composition of claim 1, wherein the first peptide component comprises a synthetic peptide synthesized from a T cell inhibitory protein.

11. An isolated nucleic acid encoding the peptide of claim 1.

12. A composition comprising the isolated nucleic acid of claim 11 and a carrier.

13. The composition of claim 11, wherein the carrier is a pharmaceutically acceptable carrier.

14. The composition of claim 13, wherein the pharmaceutically acceptable carrier is alum.

15. An isolated host cell containing the nucleic acid of claim 11.

16. A composition containing the nucleic acid of claim 13 and a carrier.

17. The composition of claim 16, wherein the carrier is a pharmaceutically acceptable carrier.

18. The composition of claim 17, wherein the pharmaceutically acceptable carrier is alum.

19. The composition of claim 18, further comprising an effective amount of a cytokine and/or a co-stimulatory molecule.

20. A method for raising immune serum comprising delivering to an animal an immunogen of one any of claims 1, 2, 7, 8, 9 and 10.

21. The method of claim 20, wherein the immunogen is delivered as a composition comprising the immunogen.

22. The method of claim 21, wherein the immunogen is delivered as an isolated nucleic acid encoding the immunogen.

23. Isolated antibody serum raised by the method of claim 20.

24. The method of claim 20, wherein the animal expresses a human immunoglobulin gene.

25. The method of claim 24, wherein the human immunoglobulin gene is selected from the group consisting of IgA1, IgaA2, IgM, IgD, IgG1, IgG2, IgG3, and IgG4.

26. A method for producing a hybridoma cell line comprising fusing antibody producing cells isolated from an animal immunized with the immunogen of claim 1 with at least one myeloma cell, thereby producing the hybridoma cell line specific for the B cell epitope of said peptide.

27. The method of claim 26, wherein the antibody producing cells are isolated from a lymph node, spleen or other lymphoid organ.

28. A hybridoma cell line produced by the method of claim 26.

29. A monoclonal antibody produced by the hybridoma cell line of claim 28.

30. A method for producing a hybridoma cell line that produces a monoclonal antibody to a T cell inhibitory molecule, the method comprising
a) contacting in vitro, a lymphoid cell isolated from an animal immunized with a synthetic peptide, wherein the synthetic peptide comprises a T cell inhibitory molecule that contains a MHC Class II binding motif and a B cell epitope, with an effective amount of a composition comprising a T cell stimulation factor and with an effective amount of T cells that specifically recognize and bind the T cell inhibitory molecule that contains a MHC Class II binding motif to produce an antibody producing lymphoid cell; and
b) fusing the cell of step a) with at least one myeloma cell, to produce a hybridoma cell line that produces a monoclonal antibody to a T cell inhibitory molecule.

31. The method of claim 30, wherein the animal is a humanized animal.

32. The method of claim 31, wherein the humanized animal is a humanized rat or murine.

33. The method of claim 30, wherein the peptide of the T cell inhibitory molecule comprises an extended peptide that is altered to create a MHC Class II binding motif where none previously existed or the native amino acid sequence of the extended peptide.

34. The method of claim 30, wherein the animal from which the lymphoid cell has been isolated expresses MHC Class II gene that may be associated with an identifiable MHC Class II binding motif.

35. The method of claim 30, wherein the extended peptide was delivered to the animal alone or with an adjuvant.

36. The method of claim 30, wherein the T cell inhibitory molecule comprises a peptide selected from the group consisting of a death domain receptor, a growth hormone receptor, a growth hormone and a co-stimulatory receptor or biologically active fragments thereof having the same or similar biological activity as full length protein.

37. The method of claim 30, wherein the T cell inhibitory molecule is selected from the group consisting of CD25, CD26, CD30, CD49a, CD69, CD70, CD83, CD87, CD96, CD97, CDw108, and CD109.

38. The method of claim 36, wherein the death domain receptor is selected from the group consisting of CD95 (Fas, Apo1), TR3 (DR3, Apo3, WSL-1, TRAMP, LARD), DR4, DR5 (Apo2, TRAU-R2, TRICK, KILLER) DR6, TNFR1 and p55 (CD120a).

39. The method of claim 36, wherein the growth hormone receptor is selected from the group consisting of: CD25 (IL-2 R alpha chain), CD 119 (IFN gamma, alpha chain), CD121a (IL-1R, type I), CD121b (IL-1R, type II), CD122 (IL-2R, beta chain), CD123 (IL-3R, alpha chain), CD124 (IL-4R), CD126 (IL-6R, alpha chain), CD127 (IL-7R, alpha chain) and CD132 (common gamma chain IL-2R/4R/7R/9R/15R).

40. The method of claim 36, wherein the growth hormone is selected from the group consisting of: IL-1alpha, IL-1beta, IL-2, IL-3, IL-4, IL-5, IL-6, IL-7, IL-8, IL-9, IL-10, IL-12, IL-13, IL-14, IL-15, IL-16, IL-17 and IL-18.

41. The method of claim 37, wherein the co-stimulatory receptor is selected from the group consisting of: CD28, CD80 (B7-2), CD86 (B7-1), CD134 (OX40), CDw137 (4-1BB), CD152 (CTLA-4), CD153 (CD30 ligand) and CD154 (CD40 ligand).

42. The method of claim 30, wherein the lymphoid cell has been isolated from a lymph node, spleen or other lymphoid organ.

43. The method of claim 30, wherein the T cells are isolated from a lymph node, spleen or other lymphoid organ.

44. A hybridoma cell line produced by the method of any of claims 30, 31 or 32.

45. An monoclonal antibody produced by the hybridoma cell line of claim 44.

46. A method of inducing an immune response to an antigen comprising delivering to a subject an effective amount of the monoclonal antibody of claim 45.


SEQUENCE LISTING (6 sequences)

SEQ ID NO: 1 - protein, 9 aa, Rattus
  Amino acid # 1 is Xaa, that  is (S) serine or (T) threonine
XXXXXXXXX

SEQ ID NO: 2 - protein, 9 aa, Rattus
  Xaa is any amino acid
FXXXXXXXX

SEQ ID NO: 3 - protein, 418 aa, Homo sapiens
  DOMAIN (14)...(32): Extended peptide shown to be the B cell epitope
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGPZ

SEQ ID NO: 4 - DNA, 1250 bp, Homo sapiens
atggagcagcggccgcggggctgcgcggcggtggcggcggcgctcctcctggtgctgctgggggcccgggcccagggcggcactcgtagccccaggtgtgactgtgccggtgacttccacaagaagattggtctgttttgttgcagaggctgcccagcggggcactacctgaaggccccttgcacggagccctgcggcaactccacctgccttgtgtgtccccaagacaccttcttggcctgggagaaccaccataattctgaatgtgcccgctgccaggcctgtgatgagcaggcctcccaggtggcgctggagaactgttcagcagtggccgacacccgctgtggctgtaagccaggctggtttgtggagtgccaggtcagccaatgtgtcagcagttcacccttctactgccaaccatgcctagactgcggggccctgcaccgccacacacggctactctgttcccgcagagatacgactgtgggacctgcctgcctggcttctatgaacatggcgatggctgcgtgtcctgccccacgagcaccctggggagcgtccagagcgctgtgccgctgtctgtggctggaggcagatgttctgggtccaggtgctcctggctggccttgtggtccccctcctgcttggggccaccctgacctacacataccgccactgctggcctcacaagcccctggttactgcagatgaagctggaggaggctctgaccccaccaccggccacccatctgtcacccttggacagcgcccacacccttctagcacctcctgacagcagtgagaagatctgcaccgtccagttggtgggtaacagctggacccctggctaccccgagacccaggaggcgctctgcccgcaggtgacatggtcctgggaccagttgcccagcagagctcttggccccgctgctgcgcccacactctcgccagagtccccagccggctcgccagccatgatgctgcagccgggcccgcagctctacgacgtgatggacgcggtcccagcgcggcgctggaaggagttcgtgcgcacgctggggctgcgcgaggcagagatcgaagccgtggaggtggagatcggccgcttccgagaccagcagtacgagatgctcaagcgctggcgccagcagcagcccgcgggcctcggagccgtttacgcggccctggagcgcatggggctggacggctgcgtggaagacttgcgcagccgcctgcagcgcggcccgtga

SEQ ID NO: 5 - protein, 32 aa, Homo sapiens
  Altered TR3 peptide with MHC class II binding motif from amino acids 4 through 12.
QGGFRSPRCDCAGDFHKKIGLFCCRGCPAGHY

SEQ ID NO: 6 - protein, 27 aa, Rattus
  Altered FITC conjucated peptide containing a Lewis rat class II binding motif at the carboxyl terminus.
AGGGITYLGKAGVAIGFSGTAPVDATG